(19) United States
(12) Patent Application Publication
(10) Pub. No.: US 2010/0256136 A1
(43) Pub. Date: Oct. 7, 2010

(54) Method for Enhancing Pdt Efficacy Using a Tyrosine Kinase Inhibitor

(76) Inventors: Ravindra K. Pandey, Williamsville, NY (US); Allan Oseroff, Buffalo, NY (US); Stephanie Pincus, Buffalo, NY (US); Janet Morgan, Buffalo, NY (US); Xiang Zheng, Quincy, MA (US); Weiguo Liu, Snyder, NY (US)

Correspondence Address: MICHAEL L. DUNN, SIMPSON & SIMPSON, PLLC, 5555 MAIN STREET, WILLIAMSVILLE, NY, 14221, US

(21) Appl. No.: 12/309,028

(22) PCT Filed: Jun. 29, 2007
(86) PCT No.: PCT/US07/15263

§ 371 Date: Apr. 19, 2010

Related U.S. Application Data
(60) Provisional application No. 60/819,773, filed on Jul. 10, 2006.

(51) Int. Cl. A61K 31/5377; A61K 31/517; A61K 31/506; A61K 31/4045; A61K 31/409; A61P 35/00

(52) U.S. Cl. 514/234.5; 514/266.4; 514/252.18; 514/414; 514/410

(57) ABSTRACT

A method for treating hyperproliferative tissue in a mammal which tissue expresses ABCG2 including the steps of: a) systemically introducing from about 100 to about 1000 mg/kg of body weight of a tyrosine kinase inhibiting compound into the mammal; b) within from about 0.5 to about 24 hours after the introducing in step a) systemically introducing from about 0.05 to about 0.5 μmol per kilogram of body weight of a tumor avid photosensitizing compound, that acts as a substrate for ABC family transport protein, ABCG2 and that has a preferential light absorbance frequency; and c) exposing the hyperproliferative tissue to light at a fluence of from about 50 to about 150 J/cm² delivered at a rate of from about 5 to about 25 mW/cm² at the light absorbance frequency. The photosensitizing compound is preferably a tetrapyrollic photosensitizer compound where the tetrapyrollic compound is a chlorin, bacteriochlorin, porphyrin, pheophorbide including pyropheophorbides, purpurinimide, or bacteriopurpurinimide and derivatives thereof; provided that, the photosensizing compound is not a meso-tetra (3-hydroxyphenyl) derivative, is not a saccharide derivative and is not a hematoporphyrin.

### CROSS-REFERENCE TO RELATED APPLICATIONS

This application claims the benefit under 35 U.S.C. §119(e) of U.S. Provisional Application No. 60/819,773, filed Jul. 10, 2006.

### STATEMENT REGARDING FEDERALLY SPONSORED RESEARCH OR DEVELOPMENT

This work was supported by the NIH (USA) Grant CA55791. The United States Government may have certain rights in this invention.

### BACKGROUND OF THE INVENTION

The ATP-binding cassette protein ABCG2(breast cancer resistance protein) effluxes some of the photosensitizers used in photodynamic therapy (PDT) against hyperproliferative tissue such as tumors, and thus reduces efficacy of photodynamic therapy (PDT) using such photosensitizers.

Photodynamic therapy (PDT) is used for the treatment of many cancers. Photosensitizers are taken up by tumor cells and then activated by light (1), generating reactive oxygen species that cause cell death by necrosis or apoptosis (2). The outcome of PDT depends on accumulation of sufficient photosensitizer in tumor cells.

Expression of ATP-binding cassette (ABC) transport proteins renders tumor cells resistant to substrate chemotherapy drugs by virtue of drug efflux (3), and the effect of these transporters on intracellular photosensitizer accumulation has been examined as a potential cause of resistance to PDT. The ABC family transport protein that has been most thoroughly investigated is ABCB1, or P-glycoprotein (Pgp), but photosensitizers were found not to be substrates for ABCB1 (4-8), nor were they substrates for ABCC1, or multidrug resistance-associated protein-1 (MRP-1) (8). In contrast, another ABC family transport protein, ABCG2, or breast cancer resistance protein (BCRP), has been found to transport some photosensitizers and to decrease intracellular photosensitizer accumulation (8). Jonker et al. demonstrated that ABCG2 knock-out mice were photosensitive because of increased protoporphyrin IX (PpIX) levels (9). Robey et al. found that pheophorbide α (Pha) is a specific substrate for ABCG2 (10), and that ABCG2 also transports pyropheophorbide-a methyl ester, chlorin e6 and 5-aminolevulinic acid (ALA)-induced PpIX, but not hematoporphyrin IX, meso-tetra (3-hydroxyphenyl) porphyrin or meso-tetra (3-hydroxyphenyl) chlorin (8).

Tyrosine kinase inhibitors (TKIs), including imatinib mesylate (Gleevec) and gefitinib (Iressa) are novel agents in cancer treatment that have been found to reverse resistance to chemotherapy drugs by blocking their efflux by ABCG2 (9,11-13).

### BRIEF DESCRIPTION OF THE SEVERAL VIEWS OF THE DRAWINGS

FIG. 1A shows a Western blot analysis gel of ABCG2 protein expression in Colo 26, RIF-1, FaDu and BCC-1 cells, with HEK-293 pcDNA and HEK-293 482R cells as negative and positive controls. Colo 26, RIF-1 and BCC-1 cells express ABCG2 at variable levels, while FaDu cells lack ABCG2 expression. The Western Blot shows that HPPH is an ABCG2 substrate.

FIG. 1B is a bar graph showing intracellular levels of HPPH, measured spectrofluorometrically, in Colo 26, RIF-1, FaDu and BCC-1 cells following HPPH uptake (OH), and one-hour efflux in HPPH-free medium at 37° C. (1 h at 37° C.) or 4° C. (1h at 4° C.) (**p<0.01 comparing cells at 37° C. and 4° C.). HEK-293 pcDNA and HEK-293 482R cells were studied as controls (*p<0.05 comparing HEK-293 482R cells at 37° C. and 4° C., and Oh uptake in HEK-293 482R vs HEK-293 pcDNA cells).

FIG. 2 shows a series of bar graphs of concentrations of various photosensitizers when used in conjunction with and without tyrosine kinase inhibitor as a modulator. Modulators increase photosensitizer accumulation in vitro in cells that express ABCG2. FIG. 2 at A is a graph showing HPPH concentrations in HEK-293 pcDNA and HEK-293 R482 cells incubated with 0.8 μM HPPH for four hours with and without 10 μM imatinib mesylate (*p<0.05 for HEK-293 R482 cells). FIG. 2 at B shows HPPH concentration in RIF-1 cells incubated with 0.8 μM HPPH with no modulator, CsA, FTC and imatinib mesylate (**p<0.01 for CsA, FTC and imatinib mesylate). FIG. 2 at C shows concentration of PpIX in Colo 26 cells incubated with 0.8 mM ALA (for PpIX) with and without imatinib mesylate (**p<0.01). FIG. 2 at D shows concentrations of BPD-MA in RIF-1 cells incubated with 0.14 μM BPD-MA with and without imatinib mesylate (**p<0.01).

FIG. 3 shows a series of line graphs of in vitro cellular survival against light exposure in the presence of various photosensitizers with and without a tyrosine kinase inhibitor as a modulator. FIG. 3 at A shows ABCG2+HEK-293 R482 cells using 0.8 μM HPPH without (), and with (◯) pretreatment with 10 μM imatinib mesylate, and ABCG2- HEK-293 pcDNA cells also using 0.8 μM HPPH without (▾), or with (Δ) pretreatment with 10 μM imatinib mesylate. FIG. 3 at B shows RIF-1 cells treated with 0.8 μM of HPPH without (), or with pretreatment with 10 μM CsA (▾), FTC (▪), imatinib mesylate (♦) or 5 μM gefitinib (▴). FIG. 3 at C shows BCC-1 cells treated with 0.4 μM of HPPH without (), or with pretreatment with 10 μM of imatinib mesylate (◯). FIG. 3 at D shows FaDu cells treated with 0.8 μM HPPH without (), or following pretreatment with 10 μM (◯) or 20 μM (▾) imatinib mesylate. FIG. 3 at E shows Colo 26 cells treated with 0.8 μM ALA without () or following pretreatment with 10 μM imatinib mesylate (◯). FIG. 3 at F shows RIF-1 cells treated with 0.14 μM BPD-MA without (), or following pretreatment with 10 μM imatinib mesylate (◯). These graphs show that the modulators increases phototoxicity.

FIG. 4A shows a graph of in vivo concentration of HPPH in tumor, muscle and skin with and without imatinib mesylate tyrosine kinase inhibitor. HPPH levels in tumor, skin and muscle of C3H/HEJCr mice bearing RIF-1 tumors treated with HPPH with and without imatinib mesylate pre-treatment (data from two experiments, each with 5 mice). In the box-and-whisker plots the dark line is the mean, the light line the median, the box top and bottom are 75^{th} and 25^{th} percentiles, and the top and bottom whiskers are 90^{th} and 10^{th} percentiles. Imatinib mesylate increases levels of HPPH in tumors.

FIG. 4B is a graph showing survival of C3H/HeJCr mice bearing RIF-1 tumors with no treatment (◯); and treated with HPPH-PDT with (▴) and without (♦) imatinib mesylate pre-treatment; and with imatinib alone, without PDT (▪). Results clearly show superior efficacy with HPPH-PDT with imatinib mesylate.

FIG. 5A shows the structures of Photofrin and HPPH-lactose.

FIG. 5B is a graph showing that efflux of HPPH-lactose (left) and Photofrin (right) in RIF-1 cells did not differ at 37° C. and 4° C.

FIG. 5C is a graph showing that survival of RIF-1 cells did not differ following treatment with 1.6 μM HPPH-Lactose with () or without (◯) pre-treatment with 10 μM imatinib mesylate, nor with 2 μg/ml Photofrin with (▾) or without (Δ) pre-treatment with 10 μM imatinib mesylate and showing that HPPH-lactose and Photofrin are not ABCG2 substrates.

### BRIEF DESCRIPTION OF THE INVENTION

The invention is a method for treating hyperproliferative tissue in a mammal which tissue expresses ABCG2 including the steps of:
a) systemically introducing from about 100 to about 1000 mg/kg of body weight of a tyrosine kinase inhibiting compound into the mammal;
b) within from about 0.5 to about 24 hours after the introducing in step a) systemically introducing from about 0.05 to about 0.5 μmol per kilogram of body weight of a tumor avid photosensitizing compound, that acts as a substrate for ABC family transport protein, ABCG2 and that has a preferential light absorbance frequency; and
c) exposing the hyperproliferative tissue to light at a fluence of from about 50 to about 150 J/cm² delivered at a rate of from about 5 to about 25 mW/cm² at the light absorbance frequency.

The photosensitizing compound is preferably a tetrapyrollic photosensitizer compound where the tetrapyrollic compound is a chlorin, bacteriochlorin, porphyrin, pheophorbide including pyropheophorbides, purpurinimide, or bacteriopurpurinimide and derivatives thereof; provided that, the photosensizing compound is not a meso-tetra (3-hydroxyphenyl) derivative, is not a saccharide derivative and is not a hematoporphyrin.

The photosensitizing compound is usually a protoporphyrin IX (PpIX), a pheophorbide α (Pha), a pyropheophorbide-a alkyl ester, a chlorin e6 or a 5-aminolevulinic acid (ALA)-induced PpIX.

### DETAILED DESCRIPTION OF THE INVENTION

ABCG2 protein is an ATP-binding cassette protein (known as a breast cancer resistance protein) that is a 655 amino acid peptide that effluxes some of the photosensitizers used in photodynamic therapy (PDT) against hyerproliferative tissue such as tumors, and thus reduces efficacy of photodynamic therapy (PDT) using such photosensitizers. This protein has been known for a number of years. Details concerning this protein can be found in Stand et al., International Journal of Biochemistry and Cell Biology 37 (2005) pp 720-725, incorporated herein by reference as background art.

As discussed above, tyrosine kinase inhibitors (TKI's) were investigated with respect to their effect upon improvement of PDT effect against tumor cell lines expressing ABCG2. While the primary TKI investigated was imatinab mesylate, it is understood that the invention includes the use of other tyrosine kinase inhibitors. Examples of such tyrosine kinase inhibitors include, but are not limited to: erlotinib, geitinib, imatinib and sunitinib. All of the foregoing are known to those skilled in the art. Erlotinib is chemically known as N-(3-ethynylphenyl)-6,7-bis(methoxyethoxy) quinazolin-4-amine. Gefitinib is chemically known as N-(3 -chloro-4-fluoro-phenyly)-7-methoxy-6(3 -morpholin-4-ylpropoxy) quinazolin-4-amine. Imatinib is chemically known as 4[(4-methyl-1-piperazininyl) methyl]-N-(4-methyl-3-[(4-(3-pyidinyl)-2-pyrimidinyl) amino)-phenyl] benzamide methane sulfonate. Sunitinib is chemically known as a 1:1 compound of hydroxybutanoic acid and N-(2-(diethylamine) ethyl-5-[(Z)-(5-fluoro-1,2-dihydro-2-oxo-3h-indol-3-ylidine) methyl-carboxamide.

The tyrosine kinase inhibiting compound may be systemically introduced by ingestion or injection.

Broadly, the photosensitizing compounds for use in accordance with the invention are those photosensitizing compounds whose cell retention is adversely affected by a tyrosine kinase, especially ABCG2, or breast cancer resistance protein (BCRP). Such photosensitizing compounds generally include tetrapyrollic photosensitizer compounds where the tetrapyrollic compound is a chlorin, bacteriochlorin, porphyrin, pheophorbides including pyropheophorbides, purpurinimide, or bacteriopurpurinimide excluding meso-tetra (3-hydroxyphenyl), and saccharide derivatives and excluding hematoporphyrins. The photosensitizing compound is usually a protoporphyrin IX (PpIX), a pheophorbide α (Pha), a pyropheophorbide-a alkyl ester, a chlorin e6 or a 5-aminolevulinic acid (ALA)-induced PpIX. The photosensitizing compound is preferably a pyropheophorbide such as HPPH.

The photosensitizing compound is commonly a tetrapyrollic pharmaceutically acceptable compound that acts as a substrate for ABC family transport protein ABCG2 and that has a preferential light absorbance frequency and that has the chemical formula:

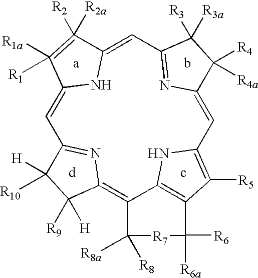

where R₁ and R₂ are each independently substituted or unsubstituted alkyl, substituted or unsubstituted alkenyl, —C(O)Rₐ or —COORₐ or —CH(CH₃)(ORₐ) or —CH(CH₃)(O(CH₂)ₙXRₐ) where Rₐ is hydrogen, substituted or unsubstituted alkyl, substituted or unsubstituted alkenyl, substituted or unsubstituted alkynyl, or substituted or unsubstituted cycloalkyl where R₂ may be

CH═CH₂, CH(OR₂₀)CH₃, C(O)Me, C(═NR₂₀)CH₃ or CH(NHR₂₀)CH₃;

where X is an aryl or heteroaryl group;

n is an integer of 0 to 6;

where R₂₀ is methyl, ethyl, butyl, heptyl, docecyl or 3,5-bis(trifluoromethyl)-benzyl; and

R₁ₐ and R₂ₐ are each independently hydrogen or substituted or unsubstituted alkyl, or together form a covalent bond;

R₃ and R₄ are each independently hydrogen or substituted or unsubstituted alkyl;

R₃ₐ and R₄ₐ are each independently hydrogen or substituted or unsubstituted alkyl, or together form a covalent bond;

R₅ is hydrogen or substituted or unsubstituted alkyl;

R₆ and R₆ₐ are each independently hydrogen or substituted or unsubstituted alkyl, or together form ═O;

R₇ is a covalent bond, alkylene, azaalkyl, or azaaraalkyl or ═NR₂₁ where R₂₁ is —CH₂X-R¹ or —YR¹ where Y is an aryl or heteroaryl group and R¹ is —H or lower alkyl;

R₈ and R₈a are each independently hydrogen or substituted or unsubstituted alkyl or together form =O;

R₉ and R₁₀ are each independently hydrogen, or substituted or unsubstituted alkyl and R₉ may be —CH₂CH₂COORₐ where Rₐ is an alkyl group;

each of Rₐ-R₁₀, when substituted, is substituted with one or more substituents each independently selected from Q, where Q is alkyl, haloalkyl, halo, pseudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue;

each Q is independently unsubstituted or is substituted with one or more substituents each independently selected from Q₁, where Q₁ is alkyl, haloalkyl, halo, pseudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or =NRₕ where Rₕ is hydrogen, alkyl, alkenyl, alkynyl, cycloalkyl, or aryl, or is an amino acid residue;

provided that, the photosensizing compound is not a meso-tetra (3-hydroxyphenyl) derivative, is not a saccharide derivative and is not a hematoporphyrin. In a preferred embodiment, R₇ is a covalent bond and the compound is a pyropheophorbide.

Usually in the method of the invention two through four doses of tyrosine kinase inhibiting compound at about 100 to about 300 mg/kg body weight is orally administered at intervals separated by from about 4 to about 12 hours in step a) and about 0.1 to about 0.3 μmol/kg of body weight of a pyropheophorbide photosensitizer is administered in step b) by injection at from about one to about three hours after completion of administration of the tyrosine kinase inhibiting compound.

Where the pyropheophorbide photosensitizer is HPPH and 24 hours after administration of the HPPH, the tumors were treated with 665 nm light from an argon ion laser-pumped dye laser with a fluence of about 50 to about 100 J/cm² delivered at a rate of about 10 to about 25 mW/cm^{2.}

The photosensizer is usually systemically administered by injection.

The invention may be illustrated by the following specific examples showing preparation of reagents for use in accordance with the invention and use thereof in determining improvement in PDT efficacy.

5-Aminolevulinic acid hydrochloride (ALA), PpIX and cyclosporin A (CsA) are known compounds and were purchased from Sigma-Aldrich (St. Louis, MO.). 2-[1-hexyloxyethyl]-2-devinyl pyropheophorbide-a (HPPH; Photochlor®), HPPH-lactose conjugate and benzoporphyrin derivative monoacid ring A (BPD-MA) were synthesized at Roswell Park Cancer Institute. Porfimer sodium (Photofrin®), a known commercially available compound, was obtained from Axcan Scandipharm, Inc. (Birmingham, AL). Imatinib mesylate (Gleevec®), a known commercially available compound, was provided by Novartis Pharmaceuticals (Basel, Switzerland) and fumitremorgin C (FTC) was provided by Dr. Susan Bates (NIH, Bethesda, MD). Gefitinib (Iressa) is a known material and was manufactured by AstraZeneca (Bristol, England).

In general known cell lines were used. FaDu human hypopharyngeal squamous cell carcinoma, RIF-1 murine radiation-induced fibrosarcoma and Colo 26 murine colon carcinoma cells were obtained from the American Type Culture Collection (ATCC; Manassas, VA). BCC-1/KMC, a human basal cell carcinoma cell line (14), was provided by Dr. Tak-Wah Wong, National Cheng Kung University Hospital, Tainan, Taiwan. HEK-293 cells transfected with either an empty pcDNA3 vector or a pcDNA3 vector containing full-length ABCG2 (HEK-293 pcDNA or HEK-293 482R) were provided by Dr. Susan Bates at the U.S. National Institute of Health, Bethesda, MD..

FaDu cells were grown in Eagle's Minimum Essential Medium (EMEM) supplemented with 10% fetal bovine serum (FBS), 200 mM L-glutamine, 1% penicillin-streptomycin, 100 mM non-essential amino acids and 1 mM MEM sodium pyruvate. RIF-1 cells were grown in MEM-α medium and BCC-1 cells and Colo 26 cells in RPMI 1640; both media were supplemented with 10% FBS, 200 mM L-glutamine and 1% penicillin-streptomycin. HEK-293 pcDNA and HEK-293 R482 cells were grown in EMEM supplemented with 10% FBS, 200 mM L-glutamine, 1% penicillin-streptomycin and 2 mg/ml G-418.

Aliquots of cell extracts were separated on 8% SDS-polyacrylamide gels by Western Blot Analysis. Protein was prepared in 30 μg quantities from all cell lines, except for HEK-293 482R cells, from which 2 μg protein were used. Proteins were transferred to Protran® membranes (Schleicher & Schuell, Riviera Beach, FL), and the membranes were reacted with antibodies to ABCB1, ABCC1 and ABCG2 (BXP-53) (Alexis Biochemicals, San Diego, CA) and β-actin (Sigma-Aldrich, St. Louis, MO). Reaction with horseradish peroxidase (HRP)-labeled secondary antibodies (ICN Biomedicals, Inc., Aurora, OH) was performed in phosphate-buffered saline (PBS) containing 0.1% Tween 20 and 5% milk. Immune complexes were visualized by an enhanced chemiluminescence (ECL) reaction (Amersham Biosciences, Piscataway, NJ). The ECL images were recorded on X-ray films with various exposure lengths.

Cells were plated in 6-well plates at a density of 3×10⁵ cells per well and incubated overnight. To study photosensitizer accumulation, cells were exposed to ABCG2 modulators including 10 μM imatinib mesylate, FTC (15) and CsA (16) for 1 hour prior to the addition of photosensitizers, which included HPPH (0.4−0.8 μM), HPPH-lactose (0.8 μM), Photofrin (2 μg/ml) and ALA (0.4-0.8 mM in 1% FCS medium). Cells were cultured for an additional 4 hours, then washed with cold culture medium and with PBS. Photosensitizer levels were determined using Solvable® (Perkin Elmer, Boston, MA) extraction (17). Briefly, the cells were solubilized in 0.5 ml Solvable® at 37° C. overnight. The Solvable® extract then was diluted 1:1 with PBS, the photosensitizer levels were determined by fluorometry, and concentrations were extrapolated from standard curves. Intracellular photosensitizer levels were normalized to intracellular protein content. To study photosensitizer efflux, cells were incubated with photosensitizer for 4 hours, then washed once with cold medium, resuspended in drug-free medium, placed at 37° C. or 4° C. for 1 hour and washed once with cold PBS. Photosensitizer levels were then determined using Solvable® extraction, as above.

Cells were plated in 96-well plates at a density of 1×10⁴ cells per well. After overnight incubation, they were exposed to ABCG2 modulators including imatinib mesylate, FTC and CsA at 10 μM and gefitinib at 5 μM, for one hour prior to the addition of photosensitizers, which included HPPH (0.4 or 0.8 μM), ALA (0.4 or 0.8 mM), BPD-MA (0.14 μM) or Photofrin (2 μg/ml), for an additional 4 hours. Cells were then irradiated with a filtered xenon arc lamp (600-700 nm) at a fluence rate of 14 mW/cm² for HPPH and BPD-MA, or with a red light (570-700 nm) at a fluence rate of 6.3 mW/cm² for ALA and Photofrin. Cell viability was evaluated by the 1,3-(4,5-dimethylthiazol-2-yl)-2,5-diphenyltetrazolium bromide (MTT) assay 48 hours after irradiation.

Eight-week-old female C3H/HeJCr mice were injected intradermally with 4×10⁵ RIF-1 tumor cells. When the tumors reached a diameter of 4 mm, groups of 5 mice received tail vein injections of 0.2 μmol/kg body weight HPPH alone or HPPH preceded by four doses of imatinib mesylate, 200 mg/kg body weight, administered by oral gavage 26, 14, 8 and 2 hours before the HPPH. To determine photosensitizer levels, samples of tumor, skin and muscle tissue were harvested 24 hours after the HPPH administration and dissolved in Solvable at 63° C. overnight. HPPH levels were measured by fluorometry as described above. In other experiments mice were administered ¹⁴C-labelled HPPH and photosensitizer levels in the harvested tissues were determined by scintillation counting. For PDT, groups of 5 tumor-bearing mice received HPPH or HPPH preceded by imatinib mesylate, as above. After 24 hours, the tumors were treated with 665 nm light from an argon ion laser-pumped dye laser (Spectra Physics, Mountain View CA) with a fluence of 72 J/cm² delivered at a rate of 14 mW/cm². Additional control mice received no treatment or imatinib mesylate alone, without HPPH. Tumors were measured every 1 to 3 days, and mice were sacrificed when tumor volumes exceeded 400 mm^{3.}

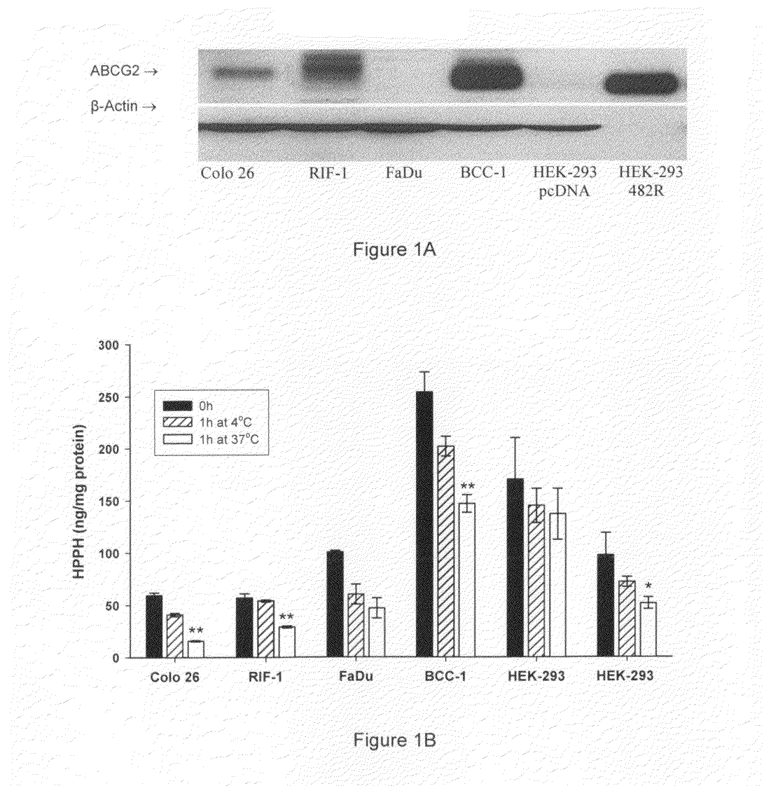

HPPH efflux was studied in cell lines with a range of levels of ABCG2 expression. Expression of ABCG2 was highest in HEK-293 R482 and BCC-1 cells, and also was high in Colo 26 and RIF-1 cells, but not in FaDu cells or HEK-293 pcDNA controls (FIG. 1A). ABCB1 and ABCC1 were not expressed in any of these cell lines (data not shown). Differences in efflux of HPPH were found among the cell lines studied (FIG. 1B). Small decreases in intracellular HPPH content were seen in all cell lines following efflux at 4° C., relative to the content following uptake (0 hour group), and these decreases were attributed to passive diffusion of HPPH from the cells. In Colo 26, RIF-1 and BCC-1 cells, intracellular HPPH levels were significantly (p<0.01) lower after efflux at 37° C., compared to 4° C., indicating energy-dependent efflux of HPPH at 37° C. in these cell lines. In contrast, HPPH content did not differ significantly following efflux at 37° C. and 4° C. in FaDu cells, which lack ABCG2 expression. Moreover, HPPH levels after uptake (0 hour group), were significantly (p<0.05) higher in ABCG2− HEK-293 pcDNA3 cells than in ABCG2+ HEK-293 R482 cells, and intracellular HPPH levels were significantly (p<0.05) higher after efflux at 4° C. than that at 37° C. in HEK-293 R482 cells, while no temperature-dependent changes in efflux of HPPH were observed in HEK293-pcDNA3 cells.

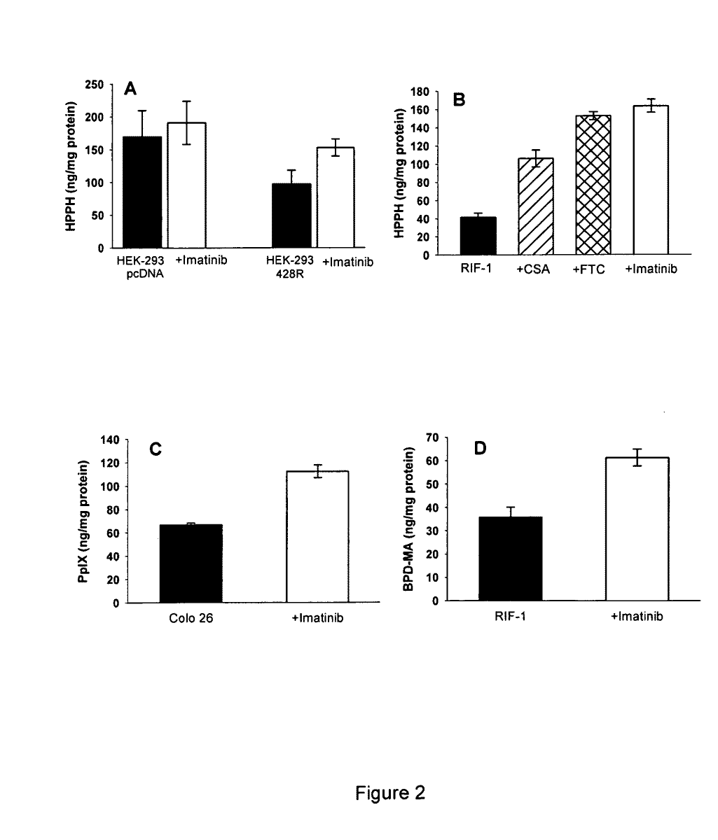

Effects of imatinib mesylate on intracellular levels of different photosensitizers were studied. Imatinib mesylate had no effect on HPPH accumulation in HEK-293 pcDNA cells, but increased intracellular HPPH levels in HEK-293 R482 cells (p<0.05) (FIG. 2A). Similar results were found for ALA/PpIX and BPD-MA (data not shown). The effects of different ABCG2 modulators on HPPH accumulation were compared in RIF-1 cells (FIG. 2B). Imatinib mesylate and FTC increased intracellular HPPH levels almost 4-fold (p<0.01), and CsA increased levels 2.5-fold (p<0.01). These effects also were demonstrated in BCC and Colo 26, which express ABCG2, but not in FaDu cells, which lack ABCG2 (data not shown). Imatinib mesylate also increased intracellular levels of two other second-generation photosensitizers, ALA-induced PpIX (FIG. 2C) and BPD-MA (FIG. 2D) in Colo 26 and RIF-1 cells, respectively, and in the other ABCG2+ cell lines, but not in FaDu cells (data not shown).

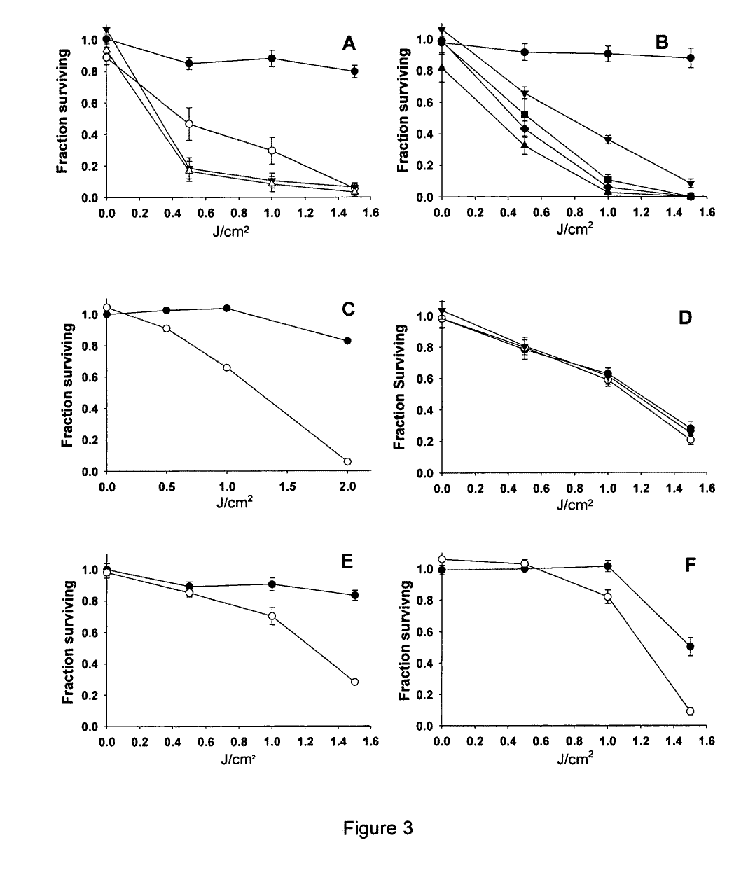

Consistent with the higher photosensitizer levels, increases in phototoxicity were observed in the presence of ABCG2 modulators in cells that expressed ABCG2. HEK-293 pcDNA cells were more sensitive to HPPH-PDT than HEK-293 R482 cells, and pretreatment with 10 μM imatinib mesylate increased phototoxicity 2- to 8-fold in HEK-293 R482 cells, depending on the light doses used, but had no effect on the sensitivity of HEK-293 pcDNA3 cells to HPPH-PDT (FIG. 3 at A). The effects of different ABCG2 modulators on HPPH phototoxicity were compared in RIF-1 cells (FIG. 3 at B). Imatinib mesylate and FTC increased HPPH-PDT phototoxicity by almost 2 logs at high light doses; gefitnib, which also blocks ABCG2 (18), was effective but had significant dark toxicity. CsA also increased phototoxicity, but to a lesser extent than the TKIs and FTC, consistent with its smaller effect on HPPH accumulation (FIG. 2 at B). Imatinib mesylate increased HPPH-PDT phototoxicity in the human basal cell carcinoma line BCC-1, which also expresses ABCG2 (FIG. 3 at C), but not in the human squamous carcinoma line FaDu (FIG. 3 at D), which lacks ABCG2 expression. Finally, imatinib mesylate also increased the phototoxicity of PpIX (shown for Colo 26 in FIG. 3 at E) and BPD-MA (shown for RIF-1 cells in FIG. 3 at F); similar enhancements were found for all of these photosensitizers in all ABCG2+ cell lines studied (data not shown).

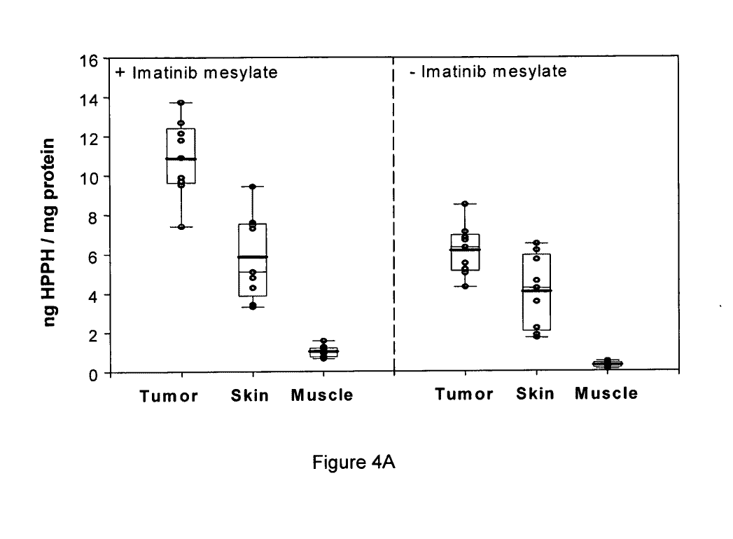

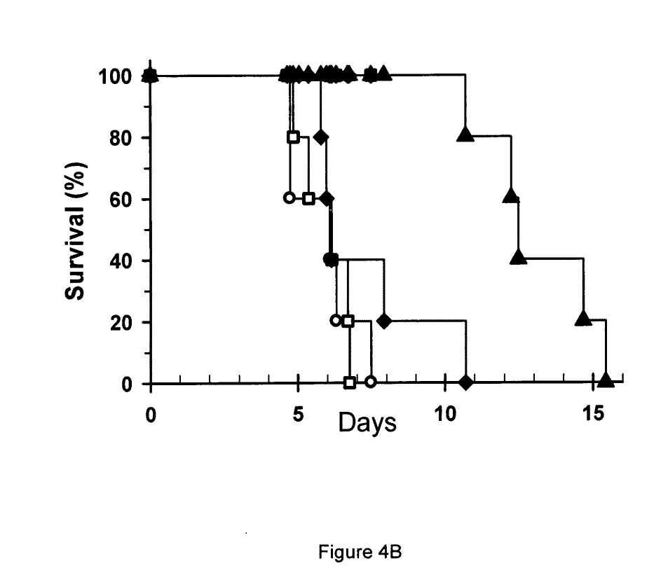

In mice bearing subcutaneous RIF-1 tumors, imatinib mesylate increased median HPPH levels in the tumors 1.8 fold (p<0.001), but had less effect on skin and muscle (FIG. 4A). The higher tumor HPPH levels correlated with enhanced in vivo PDT efficacy. Groups of mice were treated with low dose PDT using 0.2 μmol/kg HPPH followed 24 hours later by 72 J/cm² 665 nm light at 14 mW/cm². In the presence of imatinib mesylate the time for 50% of the tumors to grow to 400 mm³ doubled, from 6 to 12.5 days, compared with HPPH-PDT treatment alone (FIG. 4B). The brief course of imatinib mesylate had no anti-tumor effects and caused no observable toxicity.

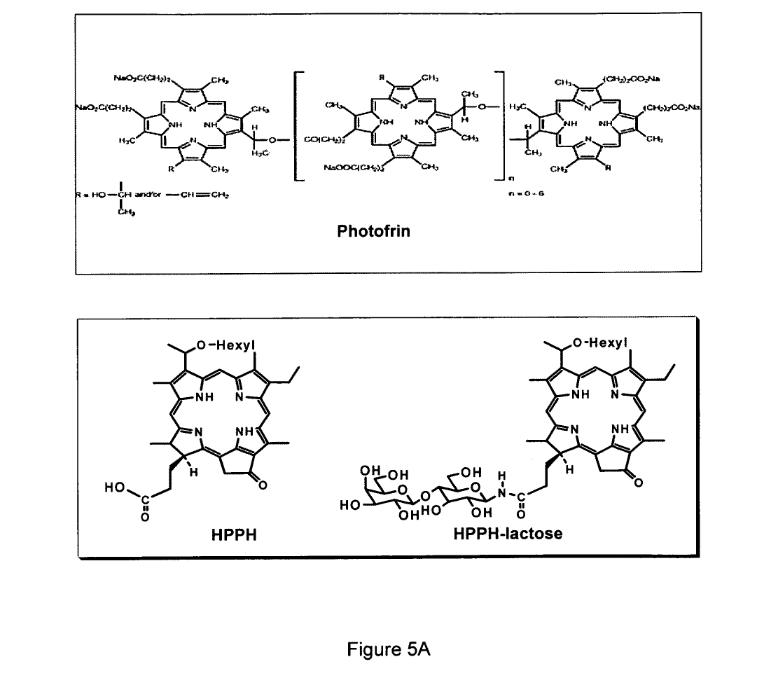

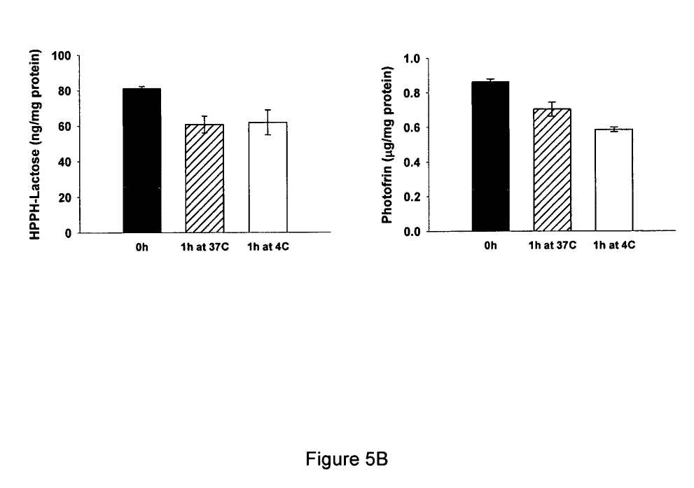

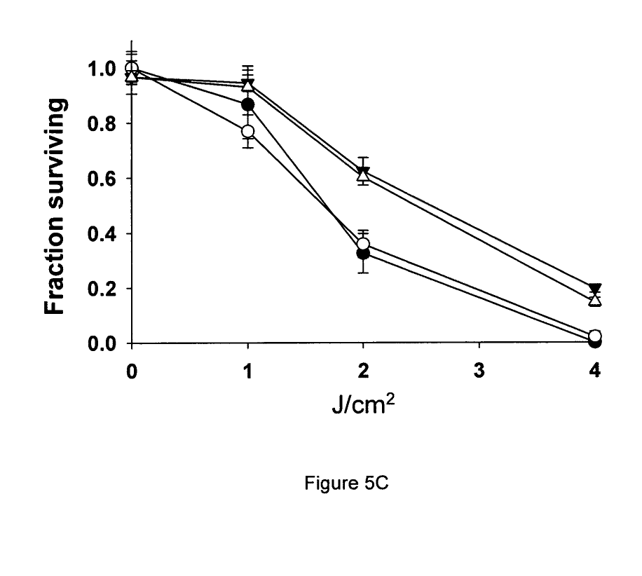

Two photosensitizers (FIG. 5A) were used to investigate whether more complex photosensitizer structures affected ABCG2-mediated transport. Temperature-dependent efflux of the first-generation multimeric agent Photofrin® (FIG. 5B, left panel) and HPPH modified by conjugation with lactose (FIG. 5B, right panel) was not found; and imatinib mesylate did not increase the phototoxicity of Photofrin-PDT or HPPH-lactose-PDT (FIG. 5C). Thus Photofrin and HPPH-lactose are not substrates for ABCG2. Similar results were obtained for other carbohydrate moieties conjugated to HPPH (data not shown).

Structure-specific active transport of three clinically used second-generation photosensitizers by ABCG2 and inhibition of ABCG2-mediated photosensitizer transport and enhancement of both in vitro and in vivo PDT through administration of the TKI imatinib mesylate have been demonstrated. TKIs increase intracellular photosensitizer accumulation and enhance phototoxicity in cells that express ABCG2. TKIs have previously been found to inhibit ABCG2-mediated transport of chemotherapy drugs and sensitize cells to chemotherapy (11-13), but the present invention provides the first demonstration that a clinically applicable TKI, imatinib mesylate, selectively increases accumulation of photosensitizer and enhances both in vitro and in vivo PDT in ABCG2+ tumor cells.

ABCG2+ cells including Colo 26, RIF-1, BCC-1 and ABCG2-transfected HEK-293 cells, exhibited decreased intracellular levels of HPPH, BPD-MA and ALA/PpIX, and resistance to PDT with these agents. In contrast, transport of these photosensitizers was not found in FaDu cells, which do not express ABCG2, or in plasmid-transfected HEK-293 cells. Note that Colo 26 cells reproducibly become ABCG2+ after about 20 passages; early passage cells are ABCG2−. Since HPPH is a derivative of pyropheophorbide-a, the results for this agent, which is in promising Phase II trials (19,20), are consistent with Robey et al.'s recent report that pyropheophorbide-a is a substrate of ABCG2 (8). The amount of HPPH transport was not directly proportional to the expression of ABCG2 measured by Western blot analysis, as exemplified by BCC-1 cells, which had higher levels of ABCG2 expression but exhibited a lesser degree of HPPH transport than the other cell lines with ABCG2 expression. Discordance between expression and function of ABCG2 has been previously demonstrated in cancer cells (21).

The mechanism(s) by which imatinib mesylate and other TKIs inhibit transport of ABCG2 substrates are being studied. Houghton et al. (12) and Jordanides et al. (22) found that imatinib mesylate inhibits ABCG2 function but is not an ABCG2 substrate (12), while Burger et al. found imatinib mesylate to be an ABCG2 substrate that inhibits pump activity by competitive inhibition (23). Ozvegy-Laczka et al. demonstrated that imatinib mesylate inhibits ABCG2 ATPase activity, possibly consistent with it not being a substrate (11). Finally, Nakanishi et al. found that imatinib decreases expression of ABCG2 protein, but not mRNA, in bcr-abl+ cells through inhibition of the PI3K-Akt pathway (24); this mechanism also might apply in malignant cells with other aberrant signaling mechanisms.

PDT acts by directly killing tumor cells, and, in many cases, by shutting down the microvasculature feeding the tumor (2). Treatment selectivity is based on higher photosensitizer levels within the target than in surrounding normal tissues, and ABCG2 expression in tumors (25,26) and on capillaries (27) can decrease both efficacy and selectivity. In addition to baseline ABCG2 expression, hypoxia, which is very common in tumors, has been found to upregulate expression of ABCG2 and to increase cell survival by decreasing intracellular accumulation of heme and other porphyrins (28). Therefore hypoxia may inhibit PDT not only because the photodynamic process requires oxygen (2), but also through ABCG2-mediated decreases in intracellular photosensitizer levels. Importantly, ABCG2+ cancer stem cells (e.g. 29, 30, 31) are expected to be relatively resistant to PDT with photosensitizers that are substrates for the ABCG2 transporter, and they may be responsible for late tumor recurrences (29,30). While ABCG2-mediated transport might be overcome by administering higher photosensitizer doses, this approach may cause unacceptable normal tissue damage. Thus, with photosensitizers that are ABCG2 substrates, inhibiting transport is likely to be a more successful approach to enhancing clinical PDT.

Administration of imatinib mesylate or other ABCG2 inhibitors in conjunction with PDT has significant potential for enhancing the efficacy of this therapeutic modality in the treatment of tumors that express ABCG2, including gastrointestinal, genitourinary, lung and head and neck cancers (25,26). Because transporter inhibition is only necessary during the interval between photosensitizer dosing and photoillumination (0.5 to 48 hours), toxicities should be minimal in relation to those associated with chronic administration of the TKI. Pump inhibition may allow lower photosensitizer doses and may improve selectivity and decrease normal tissue damage. Imatinib mesylate also may increase the levels of endogenous porphyrins in ABCG2-expressing tumors, potentially enhancing diagnosis with devices that measure endogenous fluorescence, such as Laser-Induced Fluorescence Endoscopy (LIFE) (32). Finally, it is evident that ABCG2 transport is an important, previously unconsidered factor for the design of new photosensitizers. It is not surprising that multimeric Photofrin® is not a substrate. With newer, monomeric agents, carbohydrate conjugation to a pyropheophorbide molecule blocks transport, as do the modifications in meso-tetra(3-hydroxyphenyl) porphyrin and meso-tetra(3-hydroxyphenyl) chlorin (8).

The above results show that certain second-generation photosensitizers in clinical use, especially derivatives of pyropheophorbide-a and its derivatives, are transported out of cells by ABCG2, and this effect can be abrogated by co-administration of imatinib mesylate. By increasing intracellular photosensitizer levels in ABCG2+ tumors, imatinib mesylate or other agents inhibiting ABCG2 transport may enhance efficacy and selectivity of clinical PDT.

The following references are incorporated herein by reference as background art.
1. Dolmans D E, Fukumura D, Jain R K. Photodynamic therapy for cancer. Nat Rev Cancer 2003;3:380-7.
2. Dougherty T J, Gomer C J, Henderson B W, et al. Photodynamic therapy. J Natl Cancer Inst 1998;90:889-905.
3. Gottesman M M, Fojo T, Bates S E. Multidrug resistance in cancer: role of ATP-dependent transporters. Nat Rev Cancer 2002;2:48-58.
4. Merlin J L, Gautier H, Barberi-Heyob M, Teiten M H, Guillemin F. The multidrug resistance modulator SDZ-PSC 833 potentiates the photodynamic activity of chlorin e6 independently of P-glycoprotein in multidrug resistant human breast adenocarcinoma cells. Int J Oncol 2003;22:733-9.
5. Li W, Zhang W J, Ohnishi K, Yamada I, Ohno R, Hashimoto K. 5-Aminolaevulinic acid-mediated photodynamic therapy in multidrug resistant leukemia cells. J Photochem Photobiol B 2001;60:79-86.
6. Tsai T, Hong R L, Tsai J C, Lou P J, Ling I F, Chen C T. Effect of 5-aminolevulinic acid-mediated photodynamic therapy on MCF-7 and MCF-7/ADR cells. Lasers Surg Med 2004;34:62-72.
7. Teiten M H, Bezdetnaya L, Merlin J L, et al. Effect of meta-tetra(hydroxyphenyl)chlorin (mTHPC)-mediated photodynamic therapy on sensitive and multidrug-resistant human breast cancer cells. J Photochem Photobiol B 2001;62:146-52.
8. Robey R W, Steadman K, Polgar O, Bates S E. ABCG2-mediated transport of photosensitizers: Potential impact on photodynamic therapy. Cancer Biol Ther 2005;4:187-94.
9. Jonker J W, Buitelaar M, Wagenaar E, et al. The breast cancer resistance protein protects against a major chlorophyll-derived dietary phototoxin and protoporphyria. Proc Natl Acad Sci U S A 2002;99:15649-54.
10. Robey R W, Steadman K, Polgar O, et al. Pheophorbide a is a specific probe for ABCG2 function and inhibition. Cancer Res 2004;64:1242-6.
11. Ozvegy-Laczka C, Hegedus T, et al. High-affinity interaction of tyrosine kinase inhibitors with the ABCG2 multidrug transporter. Mol Pharmacol 2004;65:1485-95.
12. Houghton P J, Germain G S, Harwood F C, et al. Imatinib mesylate is a potent inhibitor of the ABCG2 (BCRP) transporter and reverses resistance to topotecan and SN-38 in vitro. Cancer Res 2004;64:2333-7.
13. Yanase K, Tsukahara S, Asada S, Ishikawa E, Imai Y, Sugimoto Y. Gefitinib reverses breast cancer resistance protein-mediated drug resistance. Mol Cancer Ther 2004;3:1119-25.
14. Yen H T, Chiang L C, Wen K H, Tsai C C, Yu C L, Yu H S. The expression of cytokines by an established basal cell carcinoma cell line (BCC-1/KMC) compared with cultured normal keratinocytes. Arch Dermatol Res 1996;288:157-61.
15. Rabindran S K, Ross D D, Doyle L A, Yang W, Greenberger L M. Fumitremorgin C reverses multidrug resistance in cells transfected with the breast cancer resistance protein. Cancer Res 2000;60:47-50.
16. Qadir M, O'Loughlin K, Williamson N A, Fricke S M, Minderman H, Baer M R. Cyclosporin A is a broad-spectrum multidrug resistance modulator. Clin Cancer Res 2005;11:2320-2326
17. Bellnier D A, Greco W R, Parsons J C, Oseroff A R, Kuebler A, Dougherty T J. An assay for the quantitation of Photofrin in tissues and fluids. Photochem Photobiol 1997;66:237-44.
18. Nakamura Y, Oka M, Soda H, et al. Gefitinib (“Iressa”, ZD1839), an epidermal growth factor receptor tyrosine kinase inhibitor, reverses breast cancer resistance protein/ABCG2-mediated drug resistance. Cancer Res. 2005;65:1541-6.
19. Bellnier D A, Greco W R, Nava H, et al. Mild skin photosensitivity in cancer patients following injection of Photochlor (2-[1-hexyloxyethy]-2-devinyl pyropheophorbide-a; HPPH) for photodynamic therapy. Cancer Chemother Pharmacol. 2006;57:40-5.
20. Loewen G M, Pandey R, Bellnier D, et al. Endobronchial photodynamic therapy for lung cancer. Lasers Surg Med. 2006;38:364-370.
21. Suvannasankha A, Minderman H, O'Loughlin K L, et al. Breast cancer resistance protein (BCRP/MXR/ABCG2) in acute myeloid leukemia: discordance between expression and function. Leukemia 2004;18:1252-7.
22. Jordanides N E, Jorgensen H G, Holyoake T L, Mountford J C. Functional ABCG2 is over-expressed on primary CML CD34+ cells and is inhibited by imatinib mesylate. Blood 2006; Apr 20 [Epub ahead of print].
23. Burger H, van Tol H, Boersma A W, et al. Imatinib mesylate (STI571) is a substrate for the breast cancer resistance protein (BCRP)/ABCG2 drug pump. Blood 2004;104:2940-2.
24. Nakaniski T, Shiozawa K, Hassel B A, Ross D D. Complex interaction of BCRP/ABCG2 and imatinib in BCR-ABL expressing cells: BCRP-mediated resistance to imatinib is attenuated by imatinib-induced reduction of BCRP expression. Blood 2006; Mar 16 [Epub ahead of print].
25. Diestra J E, Scheffer G L, Catala I I, et al. Frequent expression of the multi-drug resistance-associated protein BCRP/MXR/ABCP/ABCG2 in human tumours detected by the BXP-21 monoclonal antibody in paraffin-embedded material. J Pathol 2002;198:213-9.
26. Kawabata S, Oka M, Soda H, Shiozawa K, et al. Expression and functional analyses of breast cancer resistance protein in lung cancer. Clin Cancer Res. 2003;9:3052-7.
27. Maliepaard M, Scheffer G L, Faneyte I F, et al. Subcellular localization and distribution of the breast cancer resistance protein transporter in normal human tissues. Cancer Res. 2001;61:3458-64.
28. Krishnamurthy P, Ross D D, Nakanishi T, et al. The stem cell marker Bcrp/ABCG2 enhances hypoxic cell survival through interactions with heme. J Biol Chem 2004;279:24218-25.
29. Houghton J, Morozov A, Smirnova I, Wang T C. Stem cells and cancer. Semin Cancer Biol. 2006 May 4; [Epub ahead of print]
30. Lam J S, Reiter R E. Stem cells in prostate and prostate cancer development. Urol Oncol. 2006;24:131-40.
31. Haraguchi N, Utsunomiya T, Inoue H, et al. Characterization of a side population of cancer cells from human gastrointestinal system. Stem Cells. 2006;24:506-13.
32. Lam S, Kennedy T, Unger M, et al. Localization of bronchial intraepithelial neoplastic lesions by fluorescence bronchoscopy. Chest 1998;113:696-702.

What is claimed is:

1. A method for treating hyperproliferative tissue in a mammal which tissue expresses ABCG2 comprising:
a) systemically introducing from about 100 to about 1000 mg/kg of body weight of a tyrosine kinase inhibiting compound into the mammal;
b) within from about 0.5 to about 24 hours after the introducing in step a) systemically introducing from about 0.05 to about 0.5 μmol per kilogram of body weight of a tumor avid photosensitizing compound, that acts as a substrate for ABC family transport protein, ABCG2 and that has a preferential light absorbance frequency; and
c) exposing the hyperproliferative tissue to light at a fluence of from about 50 to about 150 J/cm² delivered at a rate of from about 5 to about 25 mW/cm² at the light absorbance frequency.

2. The method of claim 1 where the tyrosine kinase inhibiting compound is systemically introduced by injection.

3. The method of claim 1 where the tyrosine kinase inhibiting compound is systemically introduced by ingestion.

4. The method of claim 1 where the photosensitizing compound is systemically introduced by injection.

5. The method of claim 1 where the tyrosine kinase inhibiting compound is selected from the group consisting of erlotinib, geitinib, imatinib and sunitinib.

6. The method of claim 1 where the photosensitizing compound is a tetrapyrollic photosensitizer compound where the tetrapyrollic compound is a chlorin, bacteriochlorin, porphyrin, pheophorbide including pyropheophorbides, purpurinimide, or bacteriopurpurinimide and derivatives thereof; provided that, the photosensizing compound is not a meso-tetra (3-hydroxyphenyl) derivative, is not a saccharide derivative and is not a hematoporphyrin.

7. The method of claim 5 where the photosensitizing compound is tetrapyrollic photosensitizer compound where the tetrapyrollic compound is a chlorin, bacteriochlorin, porphyrin, pheophorbides including pyropheophorbides, purpurinimide, or bacteriopurpurinimide and derivatives thereof; provided that, the photosensizing compound is not a meso-tetra (3-hydroxyphenyl) derivative, is not a saccharide derivative and is not a hematoporphyrin.

8. The method of claim 6 where the photosensitizing compound is a pyropheophorbide.

9. The method of claim 6 where the photosensitizing compound is a protoporphyrin IX (PpIX), a pheophorbide α (Pha), a pyropheophorbide-a alkyl ester, a chlorin e6 or a 5-aminolevulinic acid (ALA)-induced PpIX.

10. The method of claim 9 where the photosensitizing compound is HPPH.

11. The method of claim 1 where two through four doses of tyrosine kinase inhibiting compound at about 100 to about 300 mg/kg body weight is orally administered at intervals separated by from about 4 to about 12 hours in step a) and about 0.1 to about 0.3 μmol/kg of body weight of a pyropheophorbide photosensitizer is administered in step b) by injection at from about one to about three hours after completion of administration of the tyrosine kinase inhibiting compound.

12. The method of claim 11 where two through four doses of matinib mesylate at about 100 to about 300 mg/kg body weight is orally administered at intervals separated by from about 4 to about 12 hours in step a) and about 0.1 to about 0.3 μmol/kg of body weight of a pyropheophorbide photosensitizer is administered in step b) by injection at from about one to about three hours after completion of administration of the matinib mesylate.

13. The method of claim 12 where the pyropheophorbide photosensitizer is HPPH and 24 hours after administration of the HPPH, the tumors were treated with 665 nm light from an argon ion laser-pumped dye laser with a fluence of about 50 to about 100 J/cm² delivered at a rate of about 10 to about 25 mW/cm².

14. The method of claim 1 where the photosensitizing compound is a pharmaceutically acceptable compound that acts as a substrate for ABC family transport protein ABCG2 and that has a preferential light absorbance frequency and that has the chemical formula: 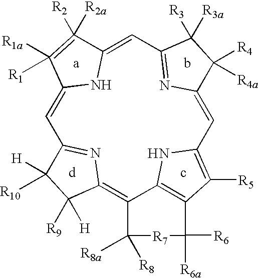
where R₁ and R₂ are each independently substituted or unsubstituted alkyl, substituted or unsubstituted alkenyl, —C(O)Rₐ or —COORₐ or —CH(CH₃)(ORₐ) or —CH(CH₃)(O(CH₂)ₙXRₐ) where Rₐ is hydrogen, substituted or unsubstituted alkyl, substituted or unsubstituted alkenyl, substituted or unsubstituted alkynyl, or substituted or unsubstituted cycloalkyl where R₂ may be CH═CH₂,CH(OR₂₀)CH₃,C(O)Me,C(═NR₂₀)CH₃ or CH(NHR₂₀)CH₃;
where X is an aryl or heteroaryl group;
n is an integer of 0 to 6;
where R₂₀ is methyl, ethyl, butyl, heptyl, docecyl or 3,5-bis(trifluoromethyl)-benzyl; and
R₁ₐ and R₂ₐ are each independently hydrogen or substituted or unsubstituted alkyl, or together form a covalent bond;
R₃ and R₄ are each independently hydrogen or substituted or unsubstituted alkyl;
R₃ₐ and R₄ₐ are each independently hydrogen or substituted or unsubstituted alkyl, or together form a covalent bond;
R₅ is hydrogen or substituted or unsubstituted alkyl;
R₆ and R₆ₐ are each independently hydrogen or substituted or unsubstituted alkyl, or together form ═O;
R₇ is a covalent bond, alkylene, azaalkyl, or azaaraalkyl or ═NR₂₁ where R₂₁ is —CH₂X-R¹ or —YR¹ where Y is an aryl or heteroaryl group and R¹ is —H or lower alkyl;
R₈ and R₈ₐ are each independently hydrogen or substituted or unsubstituted alkyl or together form ═O;
R₉ and R₁₀ are each independently hydrogen, or substituted or unsubstituted alkyl and R₉ may be —CH₂CH₂COORₐ where Rₐ is an alkyl group;
each of Rₐ-R₁₀, when substituted, is substituted with one or more substituents each independently selected from Q, where Q is alkyl, haloalkyl, halo, pseudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue;
each Q is independently unsubstituted or is substituted with one or more substituents each independently selected from Q₁, where Q₁ is alkyl, haloalkyl, halo, pseudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue; provided that, the photosensizing compound is not a meso-tetra (3-hydroxyphenyl) derivative, is not a saccharide derivative and is not a hematoporphyrin.

15. The method of claim 1 where the photosensitizing compound is a pharmaceutically acceptable compound that acts as a substrate for ABC family transport protein ABCG2 and that has a preferential light absorbance frequency and that has the chemical formula: 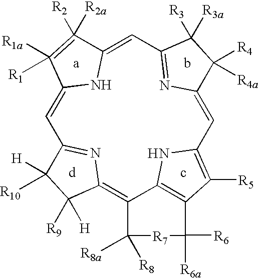
where R₁ and R₂ are each independently substituted or unsubstituted alkyl, substituted or unsubstituted alkenyl, —C(O)Rₐ or —COORₐ or —CH(CH₃)(ORₐ) or —CH(CH₃)(O(CH₂)ₙXRₐ) where Rₐ is hydrogen, substituted or unsubstituted alkyl, substituted or unsubstituted alkenyl, substituted or unsubstituted alkynyl, or substituted or unsubstituted cycloalkyl where R₂ may be CH═CH₂, CH(OR₂₀)CH₃, C(O)Me, C(═NR₂₀)CH₃ or CH(NHR₂₀)CH₃;
where X is an aryl or heteroaryl group;
n is an integer of 0 to 6;
where R₂₀ is methyl, ethyl, butyl, heptyl, docecyl or 3,5-bis(trifluoromethyl)-benzyl; and
R₁ₐ and R₂ₐ, are each independently hydrogen or substituted or unsubstituted alkyl, or together form a covalent bond;
R₃ and R₄ are each independently hydrogen or substituted or unsubstituted alkyl;
R₃ₐ and R₄ₐ are each independently hydrogen or substituted or unsubstituted alkyl, or together form a covalent bond;
R₅ is hydrogen or substituted or unsubstituted alkyl;
R₆ and R₆ₐ are each independently hydrogen or substituted or unsubstituted alkyl, or together form ═O;
R₇ is a covalent bond;
R₈ and R₈ₐ are each independently hydrogen or substituted or unsubstituted alkyl or together form ═O;
R₉ and R₁₀ are each independently hydrogen, or substituted or unsubstituted alkyl and R₉ may be —CH₂CH₂COORₐ where Rₐ is an alkyl group;
each of Rₐ-R₁₀, when substituted, is substituted with one or more substituents each independently selected from Q, where Q is alkyl, haloalkyl, halo, pseudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue;
each Q is independently unsubstituted or is substituted with one or more substituents each independently selected from Q₁, where Q₁ is alkyl, haloalkyl, halo, pseudohalo, or —COOR_{b} where R_{b} is hydrogen, alkyl, alkenyl, alkynyl, cycloalkyl, aryl, heteroaryl, araalkyl, or OR_{c} where R_{c} is hydrogen, alkyl, alkenyl, alkynyl, cycloalkyl, or aryl or CONR_{d}Rₑ where R_{d} and Rₑ are each independently hydrogen, alkyl, alkenyl, alkynyl, cycloalkyl, or aryl, or NR_{f}R_{g} where R_{f} and R_{g} are each independently hydrogen, alkyl, alkenyl, alkynyl, cycloalkyl, or aryl, or ═NRₕ where Rₕ is hydrogen, alkyl, alkenyl, alkynyl, cycloalkyl, or aryl, or is an amino acid residue; provided that, the photosensizing compound is not a meso-tetra (3-hydroxyphenyl) derivative, is not a saccharide derivative and is not a hematoporphyrin.
